(19) United States
(12) Patent Application Publication
(10) Pub. No.: US 2008/0140098 A1
(43) Pub. Date: Jun. 12, 2008

(54) Anastomosis Balloon Configurations and device

(76) Inventors: Monica Kumar, Maplewood, MN (US); Vincent G. Copa, Minnetonka, MN (US); Eric Barnum, Edina, MN (US); Suranjan Roychowdhury, Plymouth, MN (US); Kory P. Hamel, Bloomington, MN (US); Randall C. Lieser, Plymouth, MN (US); Robert L. Rykhus, Edina, MN (US); Adam L. Gullickson, Richfield, MN (US); Amy E. Geis, Shakopee, MN (US)

Correspondence Address: AMS RESEARCH CORPORATION, 10700 BREN ROAD WEST, MINNETONKA, MN, 55343, US

(21) Appl. No.: 11/985,565

(22) Filed: Nov. 15, 2007

Related U.S. Application Data
(60) Provisional application No. 60/865,869, filed on Nov. 15, 2006.

(51) Int. Cl. A61B 17/11

(52) U.S. Cl. 606/153

(57) ABSTRACT

Improved anastomosis devices having structural upgrades to increase ease of use while simultaneously increasing patient safety. The anastomosis device can include a distal treatment end and a proximal connection end. The anastomosis device has a catheter portion with a plurality of inflation lumens and a funnel portion. The funnel portion includes access ports in fluid connection to a catheter receiving aperture for attaching to the catheter portion. Also, the access ports including a drainage port, a control port and a plurality of inflation ports.

### PRIORITY CLAIM

The present application claims priority to U.S. Provisional Application Ser. No. 60/865,869, filed Nov. 15, 2006 and entitled “ANASTOMOSIS BALLOON CONFIGURATIONS AND DEVICE”, which is herein incorporated by reference in its entirety.

### FIELD OF THE INVENTION

This application relates generally to anastomosis devices for approximating and joining tissue. More particularly, the present invention is directed to structural improvements to existing anastomosis devices so as to improve upon the introduction and operation of the anastomosis device during a medical procedure.

### BACKGROUND OF THE INVENTION

Anastomosis devices and their associated procedures are generally used for connecting or re-connecting certain body tissues, e.g., as part of a surgical procedure. In typical situations, these tissues generally define a body lumen such as a blood vessel or intestinal, digestive or urinary tissue that has been severed and requires reconnection to complete a successful treatment.

Prior to the development and use of anastomosis devices, a surgeon generally performed delicate suturing operations with tiny, fine needles to reconnect these tissues. However, using these suturing techniques to connect severed body lumens was a difficult and technique-sensitive task. One factor that especially made the suturing task difficult was that in joining these body lumens, there was often a very small or limited amount of tissue to work with, such as, for example, at the urethral stump and the bladder neck. In addition, tissue such as ureters, a proximal nerve bundle and sphincter, tend to be extremely sensitive. Due to these factors, the suturing technique requires extreme care to avoid complications such as leakage, difficulty in healing or failure to heal, or specific conditions such as incontinence or impotence.

In order to overcome the difficulties associated with conventional suturing techniques, anastomosis devices utilizing a variety of tissue approximating structures to maintain severed tissue in close approximating during healing have been developed. Representative anastomosis devices include those described in U.S. Patent Publications 2005/0070938A1, 2005/0131431A1, 2006/0200178A1 and 2006/0206122A1, which are herein incorporated by reference in their entirety and are commercially available from American Medical Systems of Minnetonka, Minn. These anastomosis devices advantageously use tissue approximating structures to reconnect severed tissues during anastomosis procedures, which can both reduce the risks during the surgical procedure and also provide a significant reduction in the amount of time required to perform certain anastomosis procedures. Because the anastomosis device will typically be surgically positioned within the patient for a significant period of time (e.g., while the healing process takes place), there is a need for the device to be sufficiently strong and flexible to accommodate the various stresses to which the device may be subjected while positioned within the patient.

One representative procedure utilizing these anastomosis devices can include a radical prostatectomy procedure in which, a surgeon removes all or most of a patient's prostate. The procedure generally leaves a severed urethral stump and a severed bladder neck, which must be reconnected so as to restore proper urinary functions. Through the use of a combination of retention features including an inflation balloon and a plurality of tissue approximating structures described as extendable tines, the urethral stump and bladder neck can be aligned and retained in approximation throughout a healing period for the tissue. While the urethral stump and bladder neck forcibly hold the tissue during healing, the anastomosis device provides a drainage lumen allowing bodily fluids and other materials to pass during the healing period.

While the aforementioned anastomosis device effectively reconnects tissue during certain surgical procedures, it would be advantageous to further improve upon the existing device to increase ease of use and increased patient safety.

### SUMMARY OF THE INVENTION

The present application relates to structural improvements to anastomosis devices to make said anastomosis devices easier to use while simultaneously increasing patient safety. Generally, an anastomosis device of the present invention includes a distal treatment end and a proximal connection end. Further, the anastomosis device has a catheter portion having a plurality of inflation lumens and a funnel portion, where the funnel portion includes access ports in fluid connection to a catheter receiving aperture for attaching to the catheter portion. The access ports including a drainage port, a control port and a plurality of inflation ports.

In a first aspect, the present invention is directed to an anastomosis device including a distal treatment end and a proximal connection end, the anastomosis device having a catheter portion and a funnel portion. The funnel portion including access ports in fluid connection to a catheter receiving aperture for attaching to the catheter portion. The access ports including a drainage port, a control port and an inflation port. Additionally, the control port is connected to an actuation mechanism having a lockout mechanism that prevents patierits from manipulating the actuation mechanism.

In another aspect, an embodiment of the present invention is directed to an anastomosis device comprising a distal treatment end and a proximal connection end. The anastomosis device having a catheter portion and a funnel portion where the funnel portion includes access ports in fluid connection to a catheter receiving aperture for attaching to the catheter portion. The access ports including a drainage port, a control port and an inflation port. Further, a double balloon member is included in the distal treatment end of the device.

In another aspect, an embodiment of the present invention is directed to an anastomosis device having a distal treatment end with an inflation member and redundancy device. The anastomosis device also having a proximal connection end. The anastomosis device has a catheter portion and a funnel portion, where the funnel portion includes access ports in fluid connection to a catheter receiving aperture for attaching to the catheter portion. Further, the access ports include a drainage port, a control port and an inflation port.

In another aspect, an embodiment of the present invention is directed to a method for performing an anastomosis procedure. This method includes providing an anastomosis device with a distal treatment end and a proximal connection end, where a first balloon and a second balloon are located at the distal treatment end of the device. Also included is the step of positioning the anastomosis device within a body lumen such that the distal treatment end is proximate a treatment site, and inflating the first balloon and second balloon such that the first balloon is fully encapsulated within the second balloon.

The above summary of the invention is not intended to describe each illustrated embodiment or every implementation of the present invention. The Figures and the detailed description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of a representative embodiment of an anastomosis device of the prior art.

FIG. 2 is a cross-section of a representative embodiment of the anastomosis device of FIG. 1 being used in a radical prostatectomy procedure.

FIG. 3 is a cross-sectional view of an embodiment of an anastomosis device having a balloon-in-balloon configuration according to the present invention.

FIG. 3a is a plan view of the balloon-in-balloon configuration of FIG. 3.

FIG. 4 is a cross-sectional view of a proximal end of the anastomosis device of FIG. 3.

FIG. 5a is a plan view of an embodiment of a balloon lumen coupling according to an embodiment of the present invention.

FIG. 5b is a plan view of a balloon lumen coupling according to the prior art.

FIG. 6 is a perspective, end view of an embodiment of an anastomosis device of the present invention having an offset safety tag assembly.

FIG. 7 is a perspective, end view of the anastomosis device of FIG. 6.

FIG. 8 is a plan view of an embodiment of an anastomosis device of the present invention having hinged approximation tines.

FIG. 9 is a plan view of the anastomosis device of FIG. 8.

FIG. 10a is a plan view of an embodiment of an anastomosis device of the present invention having a webbed bladder tine configuration.

FIG. 10b is a plan view of the anastomosis device of FIG. 10a.

FIG. 10c is an end view of the anastomosis device of FIG. 10a.

FIG. 10d is an end view an alternative webbed bladder tine configuration.

FIG. 10e is a plan view of an embodiment of an anastomosis device of the present invention having an alternative webbed bladder tine configuration.

FIG. 11a is a plan view of an embodiment of an anastomosis device of the present invention having a camera bladder tine configuration.

FIG. 11b is an end view of the anastomosis device of FIG. 11a.

FIG. 12a is an end view of an embodiment of an anastomosis device of the present invention having a multiple inflation balloon configuration.

FIG. 12b is an end view of an embodiment of an anastomosis device of the present invention having a multiple inflation balloon configuration.

FIG. 13a is a plan view of an embodiment of an anastomosis device of the present invention having a reinforced half inflation balloon configuration.

FIG. 13b is an end view of the anastomosis device of FIG. 13a.

FIG. 13c is a plan view of an embodiment of an anastomosis device of the present invention having a reinforced half inflation balloon configuration.

FIG. 14 is a plan view of an embodiment of an anastomosis device of the present invention having a cross-hatched tine configuration.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

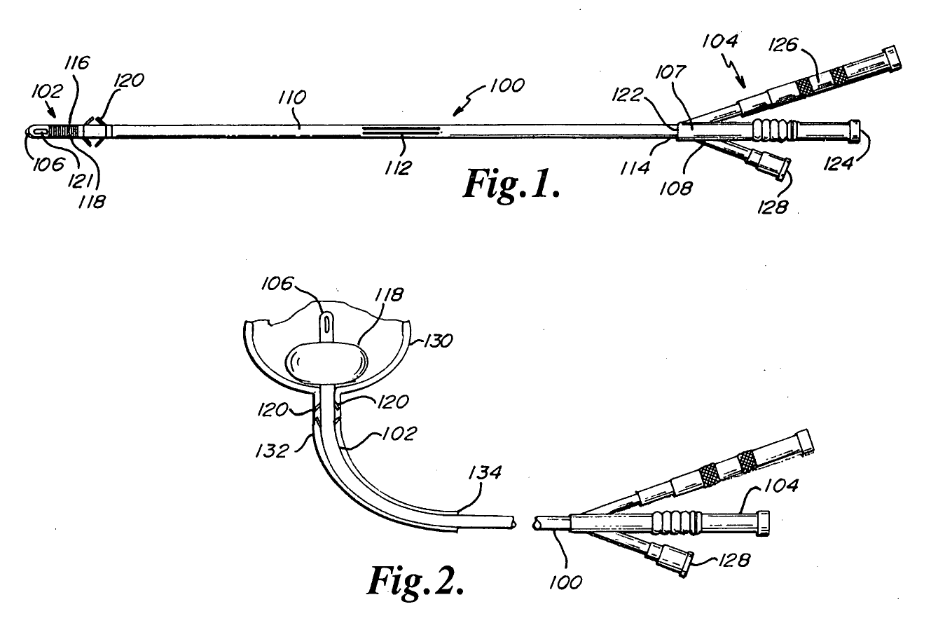

As illustrated in FIG. 1, an anastomosis device 100 of the prior art generally comprises a catheter portion 102 and a funnel portion 104 between a distal treatment end 106 and a proximal control end 108. Catheter portion 102 generally comprises a tubular body 110 defining an interior lumen 112 between the distal treatment end 106 and a proximal connection end 114. At the distal treatment end 106, catheter portion 102 can comprise a variety of retention elements 116 such as, for example, a distal inflation balloon 118 and one or more sets of distal tines 120 as well as a drainage aperture 121. Though not illustrated, it will be understood that interior lumen 112 provides space for connecting an inflation tube between the distal inflation balloon 118 and the funnel portion 104 as well as one or more guidewires for connecting the distal tines 120 with the funnel portion 104 as well as connecting a drainage lumen between the drainage aperture 121 and funnel portion 104.

Funnel portion 104 generally comprises a funnel body 107 having a catheter receiving aperture 122 and a plurality of connecting ports including a drainage port 124, a control port 126 and an inflation port 128. Drainage port 124, control port 126 and inflation port 128 include a corresponding port lumen, i.e., a drainage lumen, a control lumen and an inflation lumen operably connected to the catheter receiving aperture 122. Proximal connection end 114 is inserted into catheter receiving aperture 122 so as to operably interconnect the funnel portion 104 with the catheter portion 102.

Referring to FIG. 2, the use of anastomosis device 100 is illustrated generally with respect to connection of a patient's bladder 130 with a patient's urethra 132 such as, for example, during a radical prostatectomy. Generally, distal treatment end 106 is inserted into a urethral opening 134, through the urethra 132 and into the bladder 130. At this point, a pressurized inflation fluid can be introduced through the inflation port 128 to inflate the distal inflation balloon 118. With the distal inflation balloon 118 inflated, the bladder 130 can be pulled into approximation with the separated urethra 132 such that the distal tines 120 can be deployed to grasp and retain the bladder 130 and urethra 132 in close approximation during a healing period.

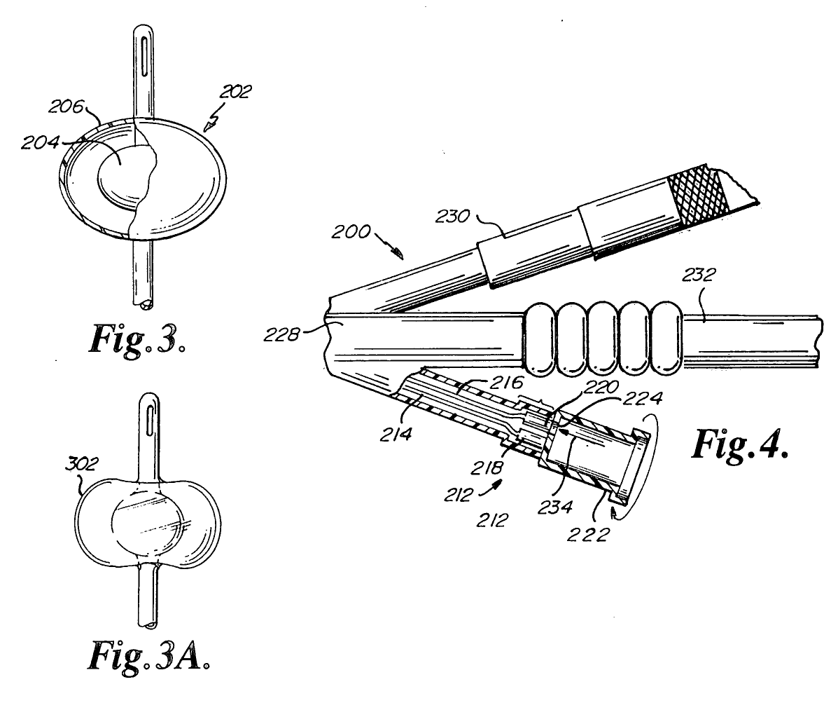

Referring now to FIGS. 3, 3a and 4, an embodiment of an anastomosis device 200 according to the present invention is illustrated. Anastomosis device 200 can substantially resemble anastomosis device 100 with the further inclusion of a balloon-in-balloon configuration 202 and a dual inflation port configuration 212. Anastomosis device 200 improves upon prior art design through by providing for redundancy and safety features should the integrity of an inflation member be compromised or otherwise damaged.

Anastomosis device 100 generally uses a balloon 118 for initial tissue approximation and to keep the drainage aperture 121 at distal treatment end 106 within bladder 130. Keeping drainage aperture 121 in position is necessary to provide for adequate urine drainage during a tissue healing period. In order to inflate the balloon 118, an inflation port 128 is provided at proximal connection end 114. With distal treatment end 106 positioned in bladder 130, inflation port 128 resides outside the body and accessible to a medical professional. In the case of balloon in balloon configuration 202, individual inflation lumens 214 and 216 and individual inflation ports 218 and 220 are provided at proximal connection end 114 so as to provide each balloon with its own lumen and inflation port.

To accommodate these features, anastomosis device 200 comprises a combined inflation port 212 with a rotating cap 222. Rotating cap 222 allows combined inflation portion 212 to selectively interface inflation ports 218, 220 with an inflation source. Rotation cap 222 allows only one inflation port 218 or 220 to be fluidly interconnected to the inflation source at any one time. In this fashion, rotation cap 222 provides for individual inflation of each inflation balloon 204 or 206 individually while keeping the number of external communicating ports to a minimum (i.e., one).

Rotating cap 222 is designed such that it has the ability to engage and disengage from each inflation port 218 or 220 without completely being detached from the anastomosis device 200. Rotating cap 222 advantageously limits the number of external communicating reports such that funnel portion 104 having three communicating ports can be utilized while still providing for individual inflation of inflation balloons 204 and 206 to create a double redundancy for the anastomosis device should one of the inflation balloons be compromised or otherwise damaged.

Referring again to FIG. 4, anastomosis device 200 includes two inflation lumens 214 and 216 linking inflation balloons 204 and 206 to the inflation ports 218 and 220. The inflation ports 218, 220 are physically positioned after catheter shaft 228 splits into an actuating lumen 230, drainage lumen 232, and inflation lumen 226. Inflation ports 218 and 220 are arranged proximate to one another within inflation lumen 226. Rotating cap 222 physically attaches to the end of inflation lumen 226. Rotatable cap 222 is only able to permit selective inflation of one inflation balloon at a time by mating with each inflation port 218, 220 individually. Further, the rotatable cap 222 is able to engage and disengage from inflation ports 218, 220 by vertical movement depicted by arrow 234, while the rotatable cap 222 remains intact with anastomosis device 200. By retaining rotatable cap 222 on anastomosis device 200, risks associated medial professionals or patients unintentionally deflating inflation balloons 204 and 206 which may lead to displacement or expulsion of the anastomosis device 200 during a healing period can be mitigated.

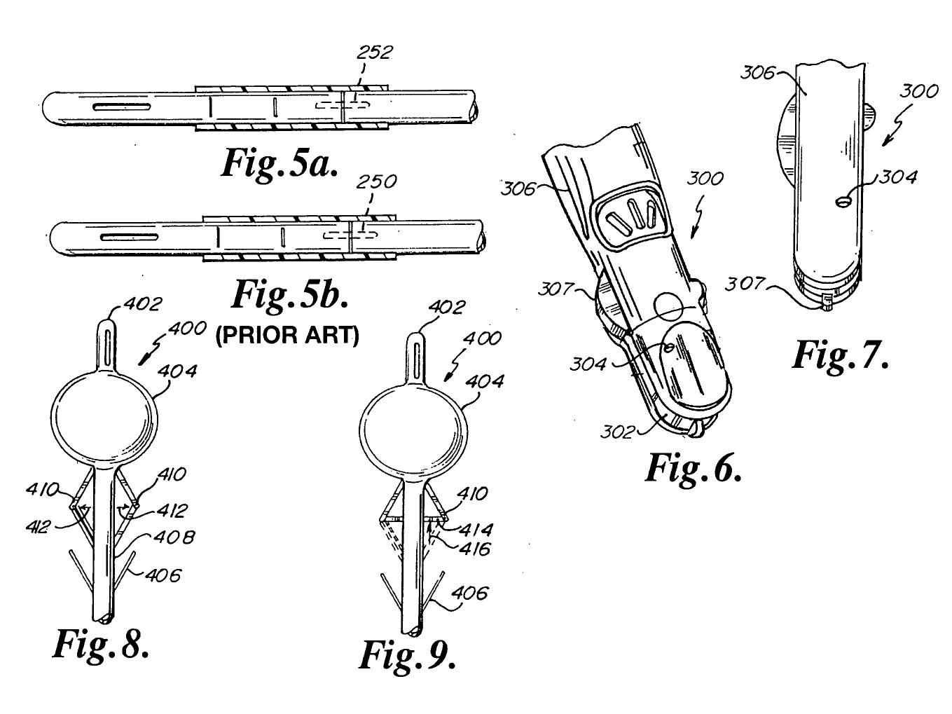

Referring now to FIGS. 5a and 5b, another embodiment is shown setting forth an improvement to the balloon lumen coupling for an anastomosis device. The purpose of the balloon lumen coupling is to provide a location where the catheter shaft may be attached to the distal tip of the anastomosis device, while continuing the open balloon lumen for inflating the balloon 112. The embodiment shown in FIG. 5a replaces the currently used stainless steel balloon lumen coupling rod 250 shown in FIG. 5b with a more pliable, non-rigid silicone tubing 252 so that the device is less susceptible to surgical tooling manipulations typically encountered during prostatectomy procedures.

The silicon tubing 252 provides for non-rigid, pliable material rather than a rigid metal rod to connect the catheter shaft to the distal tip and to continue the open balloon inflation lumen. Additionally, the device using silicon tubing 252 is less susceptible to typical surgical tooling manipulations to the device during a prostatectomy procedure.

Referring now to FIGS. 6 and 7, another embodiment for an anastomosis device 300 can substantially resemble anastomosis device 100 with the further inclusion of an offset safety tag assembly 301. The offset safety tag assembly 301 includes a looped tag member 302 made of plastic or other appropriate material, that inserted through an offset aperture 304 in the end of an actuation member 306. Actuation member 306 is generally used for operational control of distal tines 120 by way of a guidewire connection the actuation member 306 with the distal tines 120. Actuation member 306 is generally attached to anastomosis device 300 at actuation port 126.

As illustrated in FIG. 7, offset aperture 304 is physically blocked by a guidewire actuator 307 such that the tag member 302 cannot be passed through the offset aperture 304. As illustrated in FIG. 6, guidewire actuator 307 has been actuated so as to extend the distal tines 120. With guidewire actuator 307 positioned as shown in FIG. 6, offset aperture 304 is no longer blocked by the guidewire actuator 307 such that looped tag member 302 can be passed through offset aperture 304 and looped tag member 3002 can be locked and secured so as to physically prevent guidewire actuator 307 from being actuated to withdraw distal tines 120.

Offset safety tag 302 generally prevents patients from touching or manipulating the actuation member 306 and the guidewire actuator 307 specifically so as to unintentionally retract the distal tines 120 during tissue healing. Additionally, the offset aperture 304 prevents the actuation member 306 from being mistakenly locked with the distal tine 120 in a retracted disposition.

FIGS. 8 and 9 generally set forth an embodiment of an anastomosis device 400 having hinged approximation tines 410. Anastomosis device 400 generally includes a distal end 402, an inflation balloon 404, urethral tines 406, catheter shaft 408 and hinged approximation tines 410. Hinged approximation tines 410 generally provide a second retention means by which the anastomosis device 400 is able to retain its placement within the body, during a healing time period, in the event of premature rupture or deflation of inflation balloon 404. Hinged approximation tines 410 generally bow out from a catheter shaft 408 as shown by arrows 412 creating a generally a v-shape. Next, a bottom portion 414 folds inwardly, as indicated by arrows 416, thus creating a 90 degree angle with the catheter shaft 408 and providing a larger surface area for tissue contact.

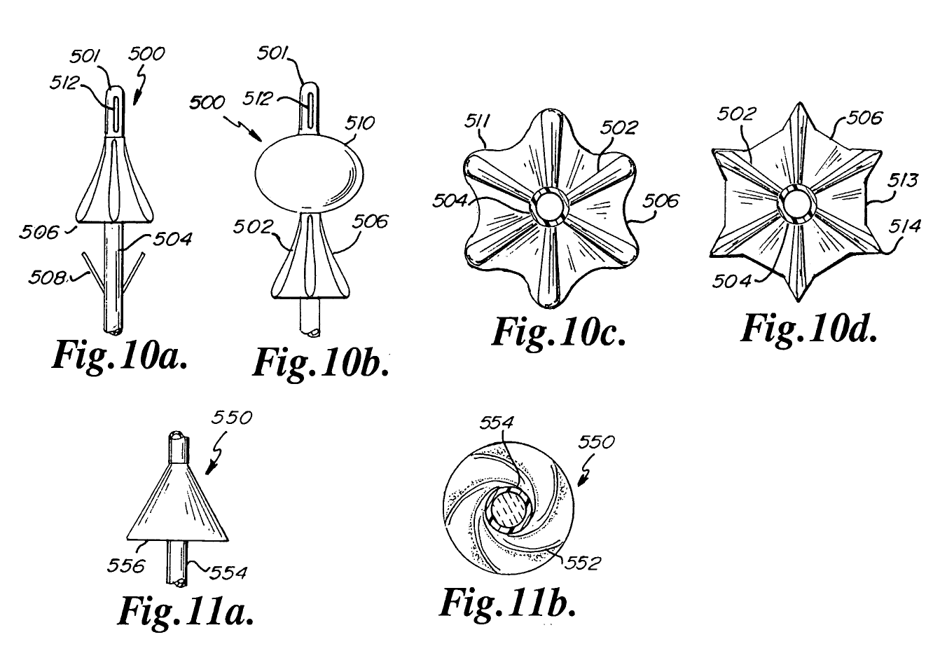

Referring now to FIGS. 10a-10e, various alternative embodiments of an anastomosis device 500 having a variety of membrane and web bladder tine configurations are illustrated. Through the use of membrane and web bladder tines, the retention capacity of anastomosis device 500 is increased in the event of a premature inflation balloon rupture or deflation during tissue healing. The membrane and web bladder tines provide additional retention strength and support to the anastomosis device 500 following their deployment.

FIG. 10a illustrates a distal end 501 of the anastomosis device 500. As shown, each anastomosis device 500 includes a plurality of bladder tines 502 which are radially mounted around catheter shaft 504 and are fixed to a sheath or web 506 along the length of the bladder tines 502. Urethral tines 508 are located proximally of the bladder tines 502 and project along the catheter shaft 504.

As illustrated in FIG. 10b, an inflation balloon 510 is in an inflated configuration and the general structure of the anastomosis device 500 can be observed. The inflation balloon 510 remains in a location medial to bladder tines 502, web 506 and drainage aperture 512.

FIG. 10c and FIG. 10d illustrate distal end views of various profile embodiments of web 506 These embodiment can comprise a star-shaped profile 511 as shown in FIG. 10c or a slightly sharper star-shaped profile 513 as shown in FIG. 10d having tips 514. FIG. 10e sets forth an embodiment wherein web 506 comprises a stent-like configuration 516 covered by a sheath 518. Regardless of the embodiment, web 506 comprises a memory shaped web which surrounds the bladder 502. Upon retraction of the bladder tines 502, the bladder tines 502 and web 506 return to their original placement surrounding bladder tines 502 and securing them against the catheter shaft 504.

FIGS. 11a and 11b set forth embodiments of a related anastomosis device 550 which utilizes camera bladder tines 552. These bladder tine members fold around a catheter shaft 554 to hold the device in place during use. A metal sheet funnel 556 is formed to make this design possible.

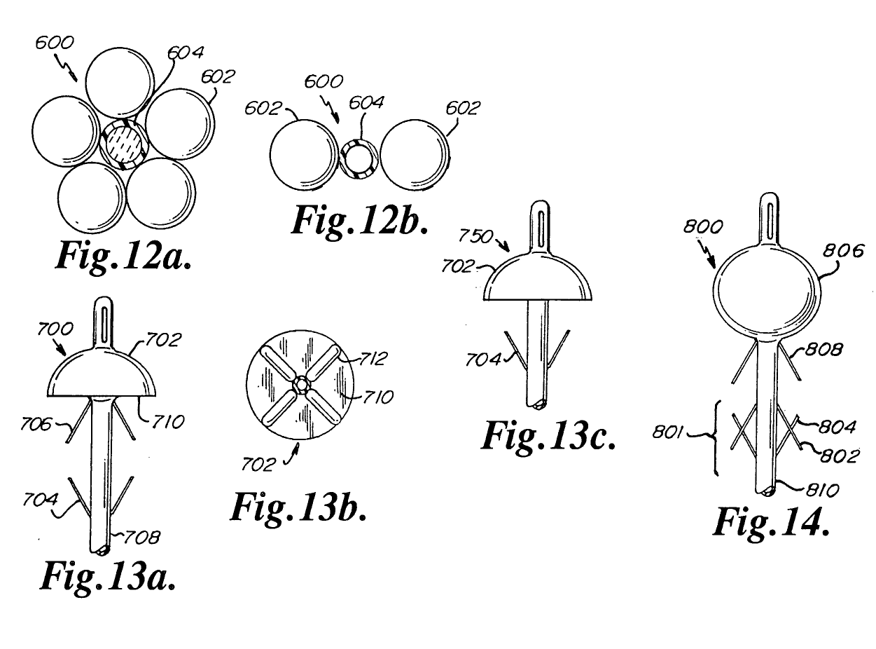

FIGS. 12a and 12b set forth embodiments of an anastomosis device 600 which utilize multiple inflation balloons 602 in another retention-redundancy design. FIG. 12a sets forth a cross-sectional view of a design 600 having multiple balloons 602 arranged around a catheter shaft 604. While five inflation balloons 602 are illustrated, any number of inflation balloons 602 can be similarly employed based the procedure performed. For example, FIG. 12b sets forth a design for anastomosis device 600 utilizing two opposing inflation balloons 602. Multiple inflation balloons 602 are employed to provide a secondary retention means by which the anastomosis device 600 retains its placement during a tissue healing period in the event of a premature balloon rupture or deflation. Multiple balloons 602 provide redundancy should one, or in some embodiments, more than one inflation balloon 602 rupture prematurely.

FIGS. 13a-13c illustrate another embodiment of an anastomosis device 700 where a reinforced half balloon member 702 is utilized. Generally, anastomosis device 700 comprises reinforced half balloon member 702, urethral tines 704, and bladder tines 706 mounted on a catheter shaft 708. Reinforced half balloon member 702 can include a reinforced bottom face 710 with ribs 712. Reinforced half balloon member 702 provides a reliable and robust balloon to prevent premature rupture or deflation of the balloon. The reinforced half balloon member 702 can in some embodiments eliminated the necessity for bladder tines 706. The reinforced bottom face 710 can be thicker than ribs 712 providing greater strength and more surface area for bladder tissue contact. In some embodiments, reinforced half balloon member 702, or any of the previously disclosed embodiments of inflation balloons, can utilized a phase changing inflation medium for inflation. The phase changing inflation medium can increase the reliability and robustness of reinforced half balloon member 702 by changing its phase (e.g., liquid to solid) once reinforced half balloon member 702 is inflated, thereby preventing the reinforced half balloon member 702 from prematurely rupturing or deflating.

FIG. 14 sets forth another embodiment of an anastomosis device 800 having cross-hatched urethral tines 801. Cross-hatched urethral tines 801 comprise an additional set of tines 802 in an opposed relation to urethral tines 804. Cross-hatched urethral tines 801 are generally located proximal an inflation balloon 806 and bladder tines 808 on a catheter shaft 810 of the anastomosis device 800. By arranging tines 802 and urethral tines 804 in an opposed configuration, a secondary means is provided by which the anastomosis device 800 is able to retain its placement once implanted in the event of a premature rupture or deflation of inflation balloon 806. Tines 802 can be deployed after the traditional sequence of deployment, further anchoring the anastomosis device 800 within the tissue to retain placement. Tines 802 generally require an actuating mechanism than that utilized for urethral tines 804 and bladder tines 808.

In addition to the disclosed and discussed embodiments, it will be understood that additional retention features can be employed in conjunction with the retention features previously described. For instance, a tapered ribbon can be employed to hold a balloon member in place, wherein the tapered ribbon provides a secondary means by which the anastomosis device is able to retain its placement once implanted in the event of a premature balloon rupture or deflation. In some embodiments, a tapered ribbon could replace or supplement the current bladder tines providing increased strength and stability and having increased contact to the bladder tissue.

In yet another embodiment, an inflation medium for the variously described inflation balloon can involve the use of an open cell foam or sponge. The open cell foam can increase the reliability and robustness of the various inflation balloons of the described anastomosis devices. The open cell foam can permanently reside within the inflation balloon in a “natural” state and would retain the inflation balloon in an inflated state. In order to deflate the inflation balloon, a vacuum can be applied to remove air from within the inflation balloon thereby allowing the open cell foam to collapse into a deflated state.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. An anastomosis device comprising:
a catheter portion having a catheter body defining a distal treatment end and a proximal connection end, the distal treatment end comprising a first inflation balloon and a second inflation balloon, wherein the first inflation balloon is fully contained within the second inflation balloon; and
a funnel portion attached to the proximal connection end of the catheter portion, the funnel portion including an interior lumen for fluidly interconnecting an inflation port on the funnel portion with the catheter body such that an inflation source coupled to the inflation port can inflate the first inflation balloon and the second inflation balloon.

2. The anastomosis device of claim 1, wherein the funnel portion further comprises a rotatable cap attached to the inflation port, the rotatable cap selectively coupling the inflation source to a first inflation lumen fluidly connected to the first inflation balloon or a second inflation lumen fluidly connected to the second inflation balloon.

3. The anastomosis device of claim 1, wherein at least one of the first inflation balloon and the second inflation balloon includes an internal inflation medium, the internal inflation medium selected from an open cell foam or sponge.

4. A method for performing an anastomosis procedure, comprising:
providing an anastomosis device with a distal treatment end including a first inflation balloon and a second inflation balloon, the first inflation balloon residing within the second inflation balloon;
positioning the distal treatment end within a body lumen such that the distal treatment end is proximate a treatment site; and
inflating the first inflation balloon; and
inflating the second inflation balloon such that the inflated first inflation balloon resides within the inflated second inflation balloon.

5. The method of claim 4, further comprising:
fabricating at least one of the first inflation balloon and the second inflation balloon to include an internal inflation medium, the internal inflation medium selected from an open cell foam or sponge.

6. An anastomosis device comprising:
a manipulation portion and a catheter portion, the catheter portion having a catheter body defining a distal treatment end having a drainage lumen, an inflation balloon and a plurality of bladder retention tines interconnected with a web material, wherein the bladder retention tines are extendable from the catheter body such that the bladder retention tines and web material define a retention profile adapted to engage a tissue interface.

7. The anastomosis device of claim 6, wherein the retention profile comprises a star-shaped retention profile.

8. An anastomosis device comprising:
a manipulation portion and a catheter portion, the catheter portion having a catheter body defining a distal treatment end, the distal treatment end having a drainage lumen, an inflation balloon, a distal set of retention tines and a proximal set of retention tines, wherein the proximal set of retention tines include a set of distal facing tines and a set of proximal facing tines, said distal facing tines and proximal facing tines overlapping to define a cross-hatched tine arrangement.

9. The anastomosis device of claim 8, wherein the set of distal facing tines and the set of proximal facing tines are independently actuatable by an actuation mechanism at the manipulation portion.

10. The anastomosis device of claim 8, wherein the distal set of retention tines comprise bladder retention tines and the proximal set of retention tines comprise urethral retention tines.

11. An anastomosis device comprising:
a manipulation portion and a catheter portion, the catheter portion having a catheter body defining a distal treatment end, the distal treatment end having a drainage lumen, an inflation balloon, a distal set of retention tines and a proximal set of retention tines, wherein the distal set of retention tines include a first tine portion and a second tine portion, the first tine portion and the second tine portion being hingedly connected such that deployment of the distal set of retention tines from the catheter body causes the first tine portion and the second tine portion to define a v-shaped retention tine configuration.

12. The anastomosis device of claim 11, wherein the distal set of retention tines comprise bladder retention tines.

13. An anastomosis device comprising:
a catheter portion having a catheter body defining a distal treatment end and a proximal connection end, the distal treatment end comprising an inflation balloon and at least one set of extendable retention tines;
a funnel portion attached to the proximal connection end of the catheter portion, the funnel portion including an interior lumen for interconnecting an actuation port on the funnel portion with the catheter body such that an actuation member can be operably coupled to the extendable retention tines so as to selectively control a deployment position of the extendable retention tines; and
a looped tag member slidingly insertable through an offset aperture located in the actuation member, the offset aperture being blocked by a guidewire actuator when the extendable retention tines are in an undeployed state and the offset aperture being open for insertion of the looped tag member when the extendable retention tines are in a deployed state.
